(19) United States
(12) Patent Application Publication
(10) Pub. No.: US 2025/0143592 A1
(43) Pub. Date: May 8, 2025

(54) BLOOD PRESSURE ESTIMATION METHOD BASED ON SPATIOTEMPORAL FEATURES OF ELECTROCARDIOGRAM AND PHOTOPLETHYSMOGRAPHY

(71) Applicant: INDUSTRY-ACADEMIC COOPERATION FOUNDATION, CHOSUN UNIVERSITY, Gwangju (KR)

(72) Inventors: Jae Hyo JUNG, Gwangju (KR); Geng Jia ZHANG, Gwangju (KR); Dae Gil CHOI, Gwangju (KR)

(73) Assignee: INDUSTRY-ACADEMIC COOPERATION FOUNDATION, CHOSUN UNIVERSITY, Gwangju (KR)

(21) Appl. No.: 18/766,168

(22) Filed: Jul. 8, 2024

(30) Foreign Application Priority Data
Nov. 7, 2023 (KR) 10-2023-0153002

(51) Int. Cl. A61B 5/021; A61B 5/00; A61B 5/352

(52) U.S. Cl. CPC A61B 5/02108; A61B 5/352; A61B 5/7267

(57) ABSTRACT

The present disclosure relates to a method of extracting spatiotemporal features of electrocardiogram (ECG) and photoplethysmography (PPG) signals corresponding to each other using a neural network and of estimating blood pressure on the basis of the spatiotemporal features. The method includes: generating a target signal of a plurality of channels by respectively combining ECG signals and PPG signals corresponding to each other; extracting a first feature composed of a plurality of channels by inputting the target signal of a plurality of channels into a 1D convolution layer; generating a channel-wise weight vector by compressing the first feature; computing a second feature composed of a plurality of channels by applying the channel-wise weight vector to the first feature; extracting a third feature by inputting the second feature into a CNN model; and determining systolic and diastolic blood pressures by inputting the third feature into an LSTM model.

### CROSS REFERENCE TO RELATED APPLICATION

The present application claims priority to Korean Patent Applications No. 10-2023-0153002, filed Nov. 7, 2023, the entire contents of which are incorporated herein for all purposes by this reference.

### BACKGROUND

### Technical Field

The present disclosure relates to a method of extracting spatiotemporal features of electrocardiogram (ECG) and photoplethysmography (PPG) signals corresponding to each other using a neural network and of estimating blood pressure on the basis of the spatiotemporal features.

### Description of the Related Art

High blood pressure is the reason for various cardiovascular diseases and cerebrovascular diseases such as a stroke, myocardial infarction, and heart failure, and according to the statistical data on cause of death of 2022 by the National Statistical Office of Korea, it was reported that four of the top 10 of causes of death are cardiocerebrovascular diseases (cardiovascular diseases, cerebrovascular diseases, diabetes, and hypertensive diseases).

Since cardiocerebrovascular diseases are usually generated by interception of blood flow due to stricture of blood vessels, it is very important to monitor blood pressure in order to prevent diseases, and for this purpose, equipment based on an oscillometric principle has been used in the related art.

Representatively, automatic blood pressure monitors based on an oscillometric principle use the method of blocking blood flowing to an arm of a user by pressing the arm through a cuff and then measuring blood pressure from the intensity of pressure applied to the cuff while e slowly removing the pressure of the cuff.

This method is generally used due to easy measurement and high accuracy, but the method has a problem that the method causes inconvenience to users because the method presses a body, a rest of at least one minute is required for repetitive measurement, and it is substantially impossible for users to perform measurement by themselves in daily life because voluminous and expensive equipment is used.

Accordingly, there is a need for a methodology that can easily monitor blood pressure in a noninvasive way.

### SUMMARY

An objective of the present disclosure is to estimate blood pressure from a combination signal of ECG and PPG using a neural network.

The objectives of the present disclosure are not limited to those described above and other objectives and advantages not stated herein may be understood through the following description and may be clear by embodiments of the present disclosure. Further, it would be easily known that the objectives and advantages of the present disclosure may be achieved by the configurations described in claims and combinations thereof.

In order to achieve the objectives, a blood pressure estimation method based on spatiotemporal features of electrocardiogram (ECG) and photoplethysmography (PPG) signals includes: generating a target signal of a plurality of channels by respectively combining ECG signals and PPG signals corresponding to each other; extracting a first feature composed of a plurality of channels by inputting the target signal of a plurality of channels into a 1D convolution layer; generating a channel-wise weight vector by compressing the first feature; computing a second feature composed of a plurality of channels by applying the channel-wise weight vector to the first feature; extracting a third feature by inputting the second feature into a CNN model; and determining systolic and diastolic blood pressures by inputting the third feature into an LSTM model.

In an embodiment, the blood pressure estimation method further includes: computing systolic and diastolic blood pressures for learning from an ambulatory blood pressure corresponding to an ECG signal for learning and a PPG signal for learning; generating a training dataset by labeling the systolic and diastolic blood pressures for learning on a signal obtained by combining the ECG signal for learning and the PPG signal for learning; and applying supervised learning to the 1D convolution layer, the CNN model, and the LSTM model using the training dataset.

In an embodiment, end-to-end learning is applied to the 1D convolution layer, the CNN model, and the LSTM model by the training dataset.

In an embodiment, the generating of a target signal includes removing noises in the ECG signal and the PPG signal by passing the ECG signal and the PPG signal through a Band Pass Filter (BPF).

In an embodiment, the generating of a target signal includes: detecting an R peak in the ECG signal; detecting a maximum peak in the PPG signal; aligning the ECG signal and the PPG signal on the basis of a time offset between the R peak and the maximum peak; and generating the target signal by combining the aligned ECG signal and PPG signal.

In an embodiment, the blood pressure estimation method includes: detecting a plurality of R peaks in an ECG signal in a unit time period; detecting a plurality of maximum peaks in a PPG signal in the unit time period; computing a time offset between an R peak and a maximum peak of a preset order of the plurality of R peaks and the plurality of maximum peaks; aligning the ECG signal and the PPG signal by applying the time offset to the ECG signal or the PPG signal; and generating the target signal by combining the aligned ECG signal and PPG signal.

In an embodiment, the generating of a target signal includes generating the target signal by time-serially connecting the PPG signal to the ECG signal.

In an embodiment, the extracting of a first feature includes extracting the first feature including temporal information by time-serially convoluting a 1D kernel to the target signal.

In an embodiment, the generating of a channel-wise weight vector includes: extracting a global feature including spatial information by squeezing the first feature; and generating the weight vector composed of elements having a value between 0 and 1 by scaling the global feature.

In an embodiment, the computing of a second feature includes computing the second feature by performing element-wise product on the channel-wise weight vector and the first feature.

In an embodiment, the CNN model extracts the third feature including spatial information from the second feature through several pairs of convolution layers and pooling layers.

In an embodiment, the LSTM model extracts a fourth feature including spatial information from the third feature.

In an embodiment, the determining of systolic and diastolic blood pressures includes: inputting the third feature into the LSTM model; inputting output of the LSTM model into a Fully Connected Layer (FCL); and determining the systolic and diastolic blood pressures in accordance with two pieces of output of the fully connected layer.

The present disclosure extracts morphological features and spatiotemporal features of ECG and PPG signals and estimates a blood pressure on the basis of the features, thereby being able to increase accuracy in estimation of a blood pressure.

Detailed effects of the present disclosure in addition to the above effects will be described with the following detailed description for accomplishing the present disclosure.

### BRIEF DESCRIPTION OF THE DRAWINGS

The above and other objectives, features and other advantages of the present disclosure will be more clearly understood from the following detailed description when taken in conjunction with the accompanying drawings, in which:

FIG. 1 is a flowchart showing a blood pressure estimation method according to an embodiment of the present disclosure;

FIG. 2 is a diagram showing removing noises from ECG and PPG signals using a band pass filter;

FIG. 3 is a diagram for explaining a process of aligning ECG and PPG signals to generate a target signal;

FIG. 4 is a diagram showing an example of a target signal obtained by combining ECG and PPG signals;

FIG. 5 is a diagram showing the structure of a blood pressure estimation neural network according to an embodiment of the present disclosure;

FIG. 6 is a flowchart showing a method of training a neural network according to an embodiment of the present disclosure;

FIG. 7 is a diagram for explaining a process of extracting features from a target signal on the basis of a weight included in a target signal;

FIG. 8 is a diagram showing an example of a CNN model and an LSTM model for feature extraction and blood pressure estimation; and

FIG. 9 is a diagram that emphasizes importantly referred parts of a target signal when a neural network estimates blood pressure.

### DETAILED DESCRIPTION

The objects, characteristics, and advantages will be described in detail below with reference to the accompanying drawings, so those skilled in the art may easily achieve the spirit of the present disclosure. However, in describing the present disclosure, detailed descriptions of well-known technologies will be omitted so as not to obscure the description of the present disclosure with unnecessary details. Hereinafter, exemplary embodiments of the present disclosure will be described with reference to accompanying drawings. The same reference numerals are used to indicate the same or similar components in the drawings.

Although terms “first”, “second”, etc. are used to describe various components in the specification, it should be noted that these components are not limited by the terms. These terms are used to discriminate one component from another component and it is apparent that a first component may be a second component unless specifically stated otherwise.

Further, when a certain configuration is disposed “over (or under)” or “on (beneath)” a component in the specification, it may mean not only that the certain configuration is disposed on the top (or bottom) of the component, but that another configuration may be interposed between the component and the certain configuration disposed on (or beneath) the component.

Further, when a certain component is “connected”, “coupled”, or “jointed” to another component in the specification, it should be understood that the components may be directly connected or jointed to each other, but another component may be “interposed” between the components or the components may be “connected”, “coupled”, or “jointed” through another component.

Further, singular forms that are used this specification are intended to include plural forms unless the context clearly indicates otherwise. In the specification, terms “configured”, “include”, or the like should not be construed as necessarily including several components or several steps described herein, in which some of the components or steps may not be included or additional components or steps may be further included.

Further, the term “A and/or B” stated in the specification means that A, B, or A and B unless specifically stated otherwise, and the term “C to D” means that C or more and D or less unless specifically stated otherwise.

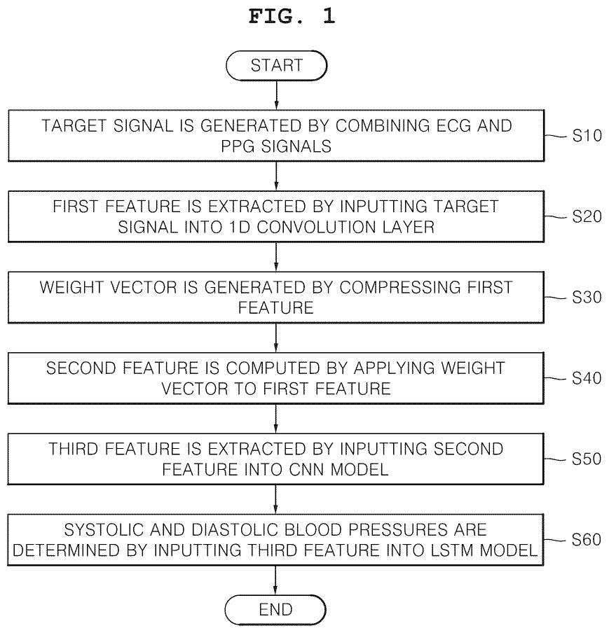

The present disclosure relates to a method of extracting spatiotemporal features of ECG and PPG signals corresponding to each other using a neural network and of estimating blood pressure on the basis of the spatiotemporal features. Hereafter, a blood pressure estimation method based on spatiotemporal features of electrocardiogram and photoplethysmography (hereafter, a blood pressure estimation method) according to an embodiment of the present disclosure is described in detail with reference to FIGS. 1 to 9.

FIG. 1 is a flowchart showing a blood pressure estimation method according to an embodiment of the present disclosure.

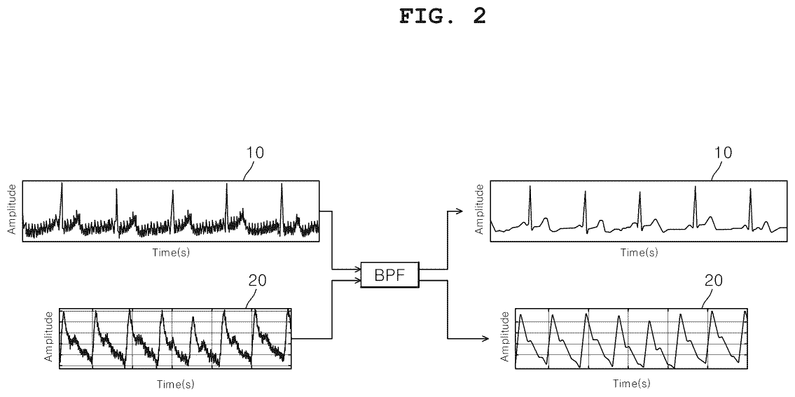

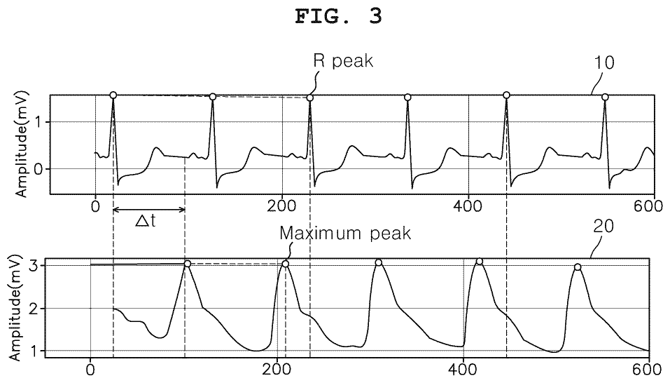

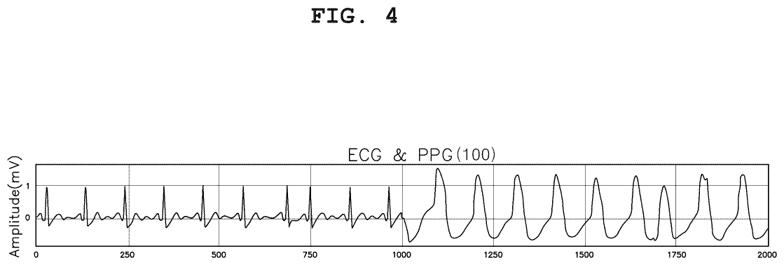

FIG. 2 is a diagram showing removing noises from ECG and PPG signals using a band pass filter, FIG. 3 is a diagram for explaining a process of aligning ECG and PPG signals to generate a target signal, and FIG. 4 is a diagram showing an example of a target signal obtained by combining ECG and PPG signals.

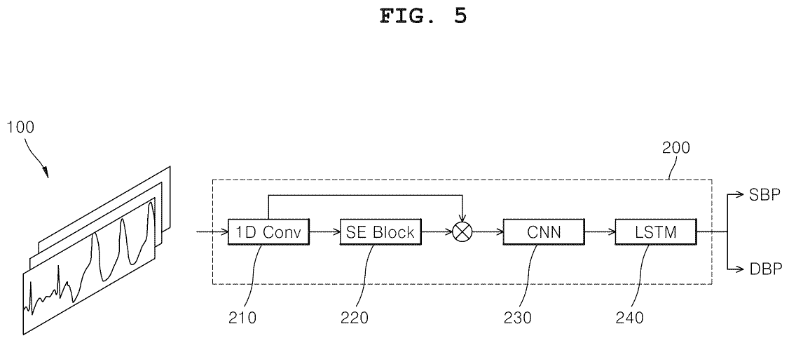

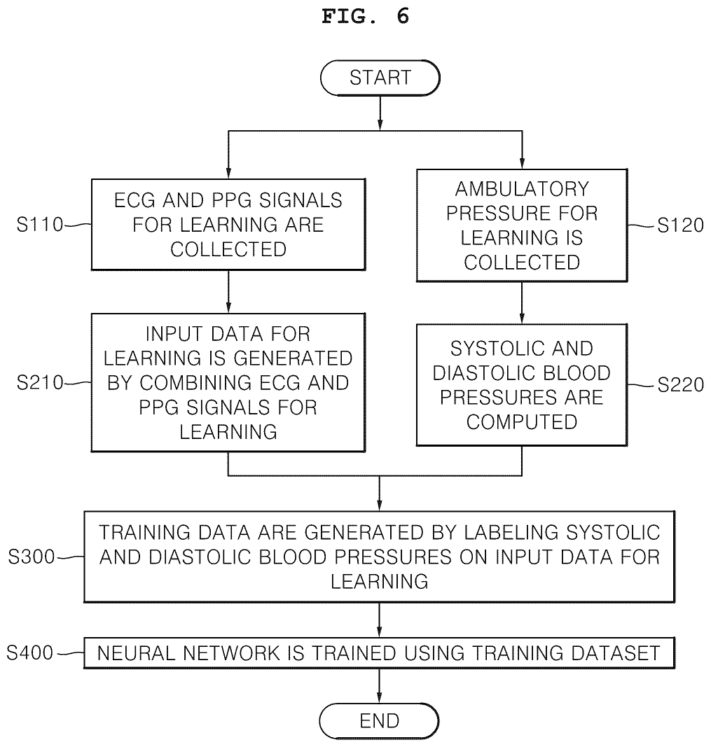

FIG. 5 is a diagram showing the structure of a neural network according to an embodiment of the present disclosure and FIG. 6 is a flowchart showing a method of training a neural network according to an embodiment of the present disclosure.

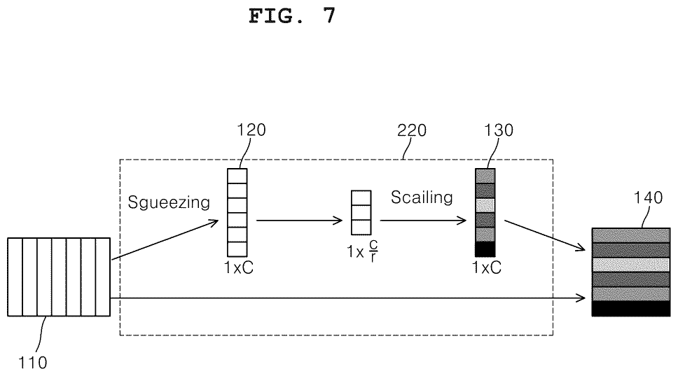

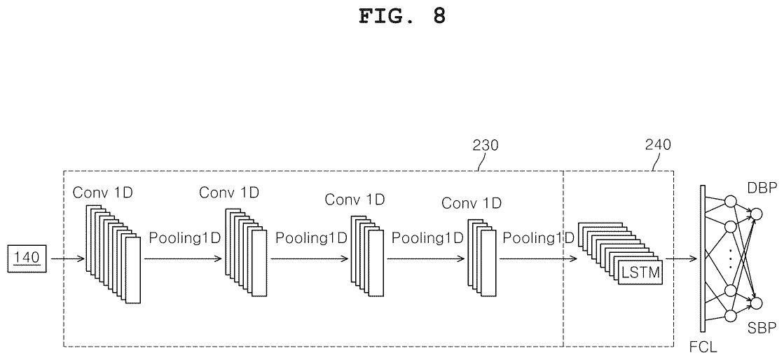

FIG. 7 is a diagram for explaining a process of extracting features from a target signal on the basis of a weight included in a target signal. FIG. 8 is a diagram showing an example of a CNN model and an LSTM model for feature extraction and blood pressure estimation.

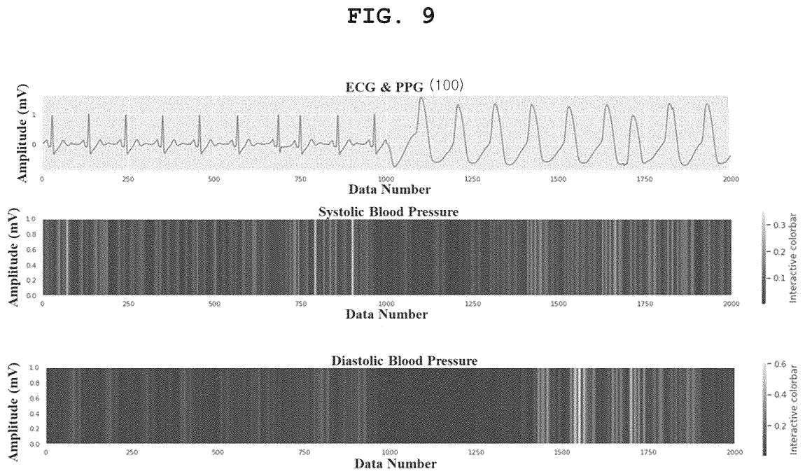

FIG. 9 is a diagram that emphasizes importantly referred parts of a target signal when a neural network estimates blood pressure.

Referring to FIG. 1, a blood pressure estimation method according to an embodiment of the present disclosure may include a step of generating a target signal by combining ECG and PPG signals (S10), a step of extracting a first feature by inputting the target signal into a 1-Dimension (1D) convolution layer (S20), a step of generating a weight vector by compressing the first feature (S30), a step of computing a second feature by applying the weight vector to the first feature (S40), a step of extracting a third feature by inputting the second feature into a Convolution Neural Network (CNN) model (S50), and a step of determining systolic and diastolic blood pressures by inputting the third feature into a Long Short-Term Memory (LSTM) model (S60).

However, the blood pressure estimation method shown in FIG. 1 is based on an embodiment, the steps of the present disclosure are not limited to the embodiment shown in FIG. 1, and if necessary, some steps may be added, changed, or removed.

Meanwhile, the steps shown in FIG. 1 may be performed by a processor, and to this end, the processor may include at least one physical element of application specific integrated circuits (ASICs), digital signal processors (DSPs), digital signal processing devices (DSPDs), programmable logic devices (PLDs), field programmable gate arrays (FPGAs), micro-controllers, and a controller.

Hereafter, the steps shown in FIG. 1 are described in detail.

A processor may generate a target signal 100 of a plurality of channels by respectively combining ECG signals 10 and PPG signals 20 corresponding to each other (S10). In this case, the term “corresponding to each other” may mean that signals are acquired in the same time period. In detail, an ECG signal 10 and a PPG signal 20 constituting one target signal 100 may be signals measured in the same time period from any one user who is a blood pressure estimation object.

The ECG signal 10, which is obtained by measuring an action current that is generated in the heart muscles in accompaniment with heartbeats. In detail, the ECG signal 10 may include a P-wave, a QRS-wave, and a T-wave and each of the waves may include information about depolarization of the atrial muscles, depolarization of the ventricular muscles, and repolarization. The ECG signal 10 may be collected from a sensor including an electrode that comes in contact with a skin, and for example, may be collected through wireless communication with a wearable device equipped with an electrode.

Meanwhile, the PPG signal 20 is obtained by measuring variation of the intensity of light emitted to a skin due to blood flow when the light is reflected and may include various items of information related to blood flow. In detail, the PPG signal 20 may have periodicity that depends on heartbeats and intensity variation that depends on various nonlinear blood flow characteristics. The PPG signal 20 may be collected from a certain sensor that can perform photoplethysmography, and for example, may be collected through wireless communication with a wearable device equipped with a photoplethysmographic sensor.

The processor can generate target signals 100 of a plurality of channels by combining ECG signals 10 measured respectively in various time periods and PPG signals 20 corresponding to the ECG signals 10. That is, the channel may mean the number of target signals 100, that is, the number of measured time periods in the present disclosure.

Meanwhile, the processor can remove noises included in the ECG signals 10 and the PPG signals 20 before generating the target signals 100.

Referring to FIG. 1, the processor according to an embodiment can remove noises by passing an ECG signal 10 and a PPG signal 20 through a band pass filter (BPF). In this case, the pass band may be freely set by a user, but a lower cutoff frequency may be set as about 0.9 [Hz] so that low physiological vibrations related to breathing and blood vessel exercises can be conserved, and a higher cutoff frequency may be set as about 10 [Hz] to remove high-frequency noises.

Further, the processor can align an ECG signal 10 and a PPG signal 20 before generating a target signal 100.

Referring to FIG. 3, in an embodiment, the processor 10 can detect an R peak in an ECG signal 10 and can detect a maximum peak in a PPG signal 20. In this case, the maximum peak may mean a peak of which the value is the maximum in one cycle of the PPG signal 20. Next, the processor can align the ECG signal 10 and the PPG signal 20 on the basis of a time offset it between the R peak and the maximum peak.

Meanwhile, this alignment operation may be performed on an ECG signal 10 in a preset unit time period rather than the entire ECG signal 10. For example, when a unit time period is set as 6 seconds, as shown in FIG. 3, the processor can detect a plurality of (in detail, six) R peaks in an ECG signal 10 for 6 seconds and can detect a plurality of (in detail, five) maximum peaks in a PPG signal 20 for 6 seconds. Next, the processor can compute a time offset Δt between the R peak and the maximum peak of a preset order, for example, the first R peak and maximum peak of the plurality of R peaks and the plurality of maximum peaks.

The processor can align the ECG signal 10 and the PPG signal 20 by applying the time offset Δt to the ECG signal 10 or the PPG signal 20. In an embodiment, as shown in FIG. 3, when a time offset Δt is determined, the processor can align two signals on the basis of the ECG signal 10 by subtracting the time offset Δt from the PPG signal 20. On the contrary, the processor may align two signals on the basis of the PPG signal 20 by adding the time offset Δt to the ECG signal 10.

Next, the processor can generate a target signal 100 by combining the aligned ECG signal 10 and PPG signal 20. In this case, combining two signals may mean combining signals measured in two different domains such that the signals are continuous in terms of time rather than combining the intensity of two signals.

In detail, referring to FIG. 4, the processor can generate a target signal by time-serially connecting an ECG signal 10 and a PPG signal aligned in a unit time period of 10 seconds. In other words, the processor can generate a target signal 100 by connecting the start point of a PPG signal 20 having a length of 10 seconds to the end of an ECG signal 10 having a length of 10 seconds. Generation of such a target signal 100 may be performed on an ECG signal 10 and a PPG signal 20 of each channel, and accordingly, the target signal 100 may also have a plurality of channels.

Meanwhile, an ECG signal 10 and a PPG signal 20 may have intensity of different scales, and in the present disclosure, normalization or scaling may not be performed on the intensity of two signals so that a neural network to be described below can learn morphological differences between an ECG signal 1 and a PPG signal 20.

Referring to FIG. 5, the processor can estimate a Systolic Blood Pressure (SBP) and a Diastolic Blood Pressure (DBP) by inputting a target signal 100 of a plurality of channels generated in accordance with the method described above into a blood pressure estimation neural network 200 and the blood pressure estimation neural network 200 may be trained in advance to perform this operation.

Hereafter, a method of training the blood pressure estimation neural network 200 is described before the operation of the present disclosure is described.

Referring to FIG. 6, a method of training the blood pressure estimation neural network 200 according to an embodiment of the present disclosure may include a step of collecting an ECG signal for learning and a PPG signal for learning (S110), a step of collecting ambulatory blood pressure for learning (S120), a step of generating input data for learning by combining the ECG signal and PPG signal for learning (S210), a step of computing systolic and diastolic blood pressures on the basis of the Ambulatory Blood Pressure (ABP) (S220), a step of generating a training dataset by labeling the systolic and diastolic blood pressures on the input data for learning (S300), and a step of training the blood pressure estimation neural network 200 using the training dataset (S400).

However, the training method shown in FIG. 6 is based on an embodiment, the steps of the present disclosure are not limited to the embodiment shown in FIG. 6, and if necessary, some steps may be added, changed, or removed.

Hereafter, the steps shown in FIG. 6 are described in detail.

The processor can collect an ECC signal for learning and a PPG signal for learning measured for the same time for the same examinee from a pre-constructed database, and an ambulatory signal (S110 and S120). In the present disclosure, the database may be a Medical Information Mart for Intensive Care (MIMIC) database that stores, for example, biological signal data of patients collected at an intensive care unit.

The processor can generate input data for learning by combining an ECG signal for learning and a PPG signal for learning (S210) in accordance with the method described in step S10, and simultaneously, can compute a systolic blood pressure and a diastolic blood pressure for learning from an ambulatory blood pressure (S220). In detail, an ambulatory blood pressure, which is obtained by measuring a blood pressure during ordinary activity, may include information about systolic and diastolic blood pressures with a white coat effect removed, and the processor can compute systolic and diastolic blood pressures for learning from the ambulatory blood pressure through various methodologies used in this technical field.

Next, the processor can generate a training dataset by labeling the systolic and diastolic blood pressures for learning on the input data for learning (S300). In other words, the processor can generate a plurality of training datasets composed of [input data for learning and systolic and diastolic blood pressures for learning] by setting systolic and diastolic blood pressures for learning as a label (Ground Truth (GT)) for input data for learning.

Next, the processor can apply supervised learning to the blood pressure estimation neural network 200 such that the blood pressure estimation neural network 200 receives a plurality of input data for learning and outputs systolic and diastolic blood pressures for learning corresponding to the input data.

In detail, the processor can input a plurality of input data for learning into the blood pressure estimation neural network 200 and can apply supervised learning to the blood pressure estimation neural network 200 such that the differences between estimated values output from the blood pressure estimation neural network 200 and the labeled systolic and diastolic blood pressures for learning become minimum. To this end, the processor can generate a first objective function proportioned to |estimated systolic blood pressure−labeled systolic blood pressure| and a second objective function proportioned to |estimated diastolic blood pressure−labeled diastolic blood pressure|, and can train the blood pressure estimation neural network 200 such that the first and second objective functions each become minimum.

Such training may be performed in an end-to-end manner. In other words, as shown in FIG. 5, the blood pressure estimation neural network 200 of the present disclosure may include a 1D convolution layer 210, a CNN model 230, and an LSTM model 240 and end-to-end supervised learning may be applied to them in accordance with setting of input and output data described above.

Hereafter, detailed operation of the blood pressure estimation neural network 200 for determining systolic and diastolic blood pressures of a target user is described and the blood pressure estimation neural network 200 to be described hereafter should be understood as having been trained in accordance with the process described above.

Referring to FIG. 5 again, the processor can extract a first feature 110 composed of a plurality of channels by inputting a target signal 100 of a plurality of channels generated in step S10 into the 1D convolution layer 210 (S20).

The target signal is defined as intensity over time, so it may be a 1-dimension signal that is expressed as the number of data sampled over time. Accordingly, the processor can embed the target signal 100 by inputting the target signal 100 into the 1D convolution layer 210. In detail, the processor can extract a first feature 110 by convoluting a 1D kernel to the target signal 100 time-serially, that is, in the order of data arranged over time. Accordingly, a temporal feature that the target signal 100 involves may be included in the first feature 110.

Next, the processor can generate a channel-wise weight vector 130 by compressing the first feature 110 (S30). As described above, since the target signal 100 is composed of a plurality of channels, the first feature 110 extracted in step S20 may also be composed of a plurality of channels. The processor can generate a channel-wise weight vector 130 that shows which channels are more important for blood pressure estimation in the first feature 110.

The operation of generating a weight vector 130 may be performed in an SE block 220 shown in FIG. 5, and hereafter, the operation of generating a weight vector 130 is described with reference to FIG. 7.

Referring to FIG. 7, the processor can extract a global feature 120 by squeezing a channel-wise first feature 110. In an embodiment, the processor can extract a 1 dimension global feature (1×C) 120 from a first feature 110 having a plurality of channels C by applying pooling, in detail, global average pooling to the channel-wise first feature 110. In this process, spatial information involved in the target signal 100 may be included in the global feature 120.

Next, the processor can generate a channel-wise weight vector 130 composed of elements having a value between 0 and 1 by recalibrating, in detail, contracting and expanding the global feature 120 in accordance with a preset reduction ratio r for considering channel-wise dependencies and by scaling the recalibrated global feature 120.

Next, the processor can compute a second feature 140 composed of a plurality of channels by applying the channel-wise weight vector 130 to the first feature 110 extracted in step S20 (S40).

Referring to FIGS. 5 and 7 again, the processor can compute a second feature 140 by performing element-wise product on the first feature 110 and the channel-wise weight vector 130 generated in the SE block 220. In accordance with this operation, parts that are more important for blood pressure estimation in the first feature 110 may be emphasized in the second feature 140.

Next, the processor can extract a third feature by inputting the second feature 140 into the CNN model 230 (S50). The CNN model 230, which is a model that embeds spatial information involved in the second feature 140, may include several pairs of convolution layer and pooling layer.

Describing in detail with reference to FIG. 8, the CNN model 230 may have a structure in which one pair of convolution layer and pooling layer is input into the next pair of convolution layer and pooling layer. In this case, the number of layers, the number of kernels, the stride, the activation function, the pooling technique, etc. may be appropriately set by users. Since the CNN model 230 has this structure, the CNN model 230 can extract a third feature including spatial information of the target signal 100 from the second feature 140.

Next, the processor can determine systolic and diastolic blood pressures by inputting the third feature into the LSTM model 240 (S60). The LSTM model 240, which is a model that embeds temporal information involved in the third feature, may have a structure that continuously refers to a past temporal feature through a cell state shared by continuous layers.

Describing in detail with reference to FIG. 8, the LSTM model 240 may include a plurality of continuous layers and may have a structure in which the output of a just previous layer is input into the next layer. In this case, all of the layers may share a cell state, whereby each layer can continuously refer to a feature extracted from a previous layer in its feature extraction operation. Since the LSTM model 240 has this structure, the LSTM model 240 can extract a fourth feature including temporal information of the target signal 100 from the third feature.

The processor can input the fourth feature into a Fully Connected Layer (FCL). The FCL can output a systolic blood pressure and a diastolic blood pressure through two nodes, respectively, and the processor can finally determine systolic and diastolic blood pressures on the basis of the output of the FCL.

FIG. 9 is a diagram showing a saliency map showing parts that the blood pressure estimation neural network 200 importantly refers to in the target signal 100. Referring to FIG. 9, it is possible to know that, in blood pressure estimation of the present disclosure, a QRS wave and an ST segment in an ECS signal 10 have a large influence, and a dicrotic notch, a systolic peak, and a diastolic decay in a PPG signal 20 have a large influence. Further, it is possible to know that the PPG signal 20 has significance more than the ECG signal in estimation of a systolic blood pressure.

As described above, the present disclosure extracts morphological features and spatiotemporal features of ECG and PPG signals 10 and 20 and estimates a blood pressure on the basis of the features, thereby being able to increase accuracy in estimation of a blood pressure.

Although the present disclosure was described with reference to the exemplary drawings, it is apparent that the present disclosure is not limited to the embodiments and drawings in the specification and may be modified in various ways by those skilled in the art within the range of the spirit of the present disclosure. Further, even though the operation effects according to the configuration of the present disclosure were not clearly described with the above description of embodiments of the present disclosure, it is apparent that effects that can be expected from the configuration should be also admitted.

What is claimed is:

1. A blood pressure estimation method based on spatiotemporal features of electrocardiogram (ECG) and photoplethysmography (PPG) signals, the blood pressure estimation method comprising:
generating, by a processor, a target signal of a plurality of channels by respectively combining ECG signals and PPG signals corresponding to each other;
extracting, by a processor, a first feature composed of a plurality of channels by inputting the target signal of a plurality of channels into a 1D convolution layer;
generating, by a processor, a channel-wise weight vector by compressing the first feature;
computing, by a processor, a second feature composed of a plurality of channels by applying the channel-wise weight vector to the first feature;
extracting, by a processor, a third feature by inputting the second feature into a CNN model; and
determining, by a processor, systolic and diastolic blood pressures by inputting the third feature into an LSTM model.

2. The blood pressure estimation method of claim 1, further comprising:
computing, by a processor, systolic and diastolic blood pressures for learning from an ambulatory blood pressure corresponding to an ECG signal for learning and a PPG signal for learning;
generating, by a processor, a training dataset by labeling the systolic and diastolic blood pressures for learning on a signal obtained by combining the ECG signal for learning and the PPG signal for learning; and
applying, by a processor, supervised learning to the 1D convolution layer, the CNN model, and the LSTM model using the training dataset.

3. The blood pressure estimation method of claim 2, wherein end-to-end learning is applied to the 1D convolution layer, the CNN model, and the LSTM model by the training dataset.

4. The blood pressure estimation method of claim 1, wherein the generating of a target signal includes removing noises in the ECG signal and the PPG signal by passing the ECG signal and the PPG signal through a Band Pass Filter (BPF).

5. The blood pressure estimation method of claim 1, wherein the generating of a target signal includes:
detecting an R peak in the ECG signal;
detecting a maximum peak in the PPG signal;
aligning the ECG signal and the PPG signal on the basis of a time offset between the R peak and the maximum peak; and
generating the target signal by combining the aligned ECG signal and PPG signal.

6. The blood pressure estimation method of claim 1, wherein the generating of a target signal includes:
detecting a plurality of R peaks in an ECG signal in a unit time period;
detecting a plurality of maximum peaks in a PPG signal in the unit time period;
computing a time offset between an R peak and a maximum peak of a preset order of the plurality of R peaks and the plurality of maximum peaks;
aligning the ECG signal and the PPG signal by applying the time offset to the ECG signal or the PPG signal; and
generating the target signal by combining the aligned ECG signal and PPG signal.

7. The blood pressure estimation method of claim 1, wherein the generating of a target signal includes generating the target signal by time-serially connecting the PPG signal to the ECG signal.

8. The blood pressure estimation method of claim 1, wherein the extracting of a first feature includes extracting the first feature including temporal information by time-serially convoluting a 1D kernel to the target signal.

9. The blood pressure estimation method of claim 1, wherein the generating of a channel-wise weight vector includes:
extracting a global feature including spatial information by squeezing the first feature; and
generating the weight vector composed of elements having a value between 0 and 1 by scaling the global feature.

10. The blood pressure estimation method of claim 1, wherein the computing of a second feature includes computing the second feature by performing element-wise product on the channel-wise weight vector and the first feature.

11. The blood pressure estimation method of claim 1, wherein the CNN model extracts the third feature including spatial information from the second feature through several pairs of convolution layers and pooling layers.

12. The blood pressure estimation method of claim 1, wherein the LSTM model extracts a fourth feature including spatial information from the third feature.

13. The blood pressure estimation method of claim 1, wherein the determining of systolic and diastolic blood pressures includes:
inputting the third feature into the LSTM model;
inputting output of the LSTM model into a Fully Connected Layer (FCL); and
determining the systolic and diastolic blood pressures in accordance with two pieces of output of the fully connected layer.
